(19) United States
(12) Patent Application Publication
(10) Pub. No.: US 2007/0156099 A1
(43) Pub. Date: Jul. 5, 2007

(54) Syringe case configured for aiding prescribed delivery of a syringe-injected medication

(76) Inventor: Jerald Lawayne Fowler, Lubbock, TX (US)

Correspondence Address: David O. Simmons;Galasso & Associates LP, P.O. Box 26503, Austin, TX, 78755-0503, US

(21) Appl. No.: 11/321,804

(22) Filed: Dec. 29, 2005

(51) Int. Cl. A61M 5/00; A61M 3/00

(52) U.S. Cl. 604/189

(57) ABSTRACT

A syringe case comprises a first structural body and a second structural body. The first structural body includes a syringe retaining structure and a medication container retaining structure. The syringe retaining structure and the medication container retaining structure reside within an interior space of the first structural body. The syringe retaining structure is configured for holding a plurality of syringe such that a longitudinal axis of each syringe extends generally parallel with a longitudinal reference axis. The medication container retaining structure is configured for holding a medication container in a manner such that a longitudinal axis of the medication container extends generally perpendicular to the longitudinal reference axis. The second structural body is movably attached to the first structural body for enabling the structural bodies to be moved between an orientation providing a generally enclosed space between the structural bodies and an orientation exposing the interior space of the first structural body.

### FIELD OF THE DISCLOSURE

The disclosures made herein relate generally to carrying cases for syringes and, more particularly, to syringe cases configured for aiding prescribed delivery of a syringe-injected medication.

### BACKGROUND

Persons having diabetes (i.e., a diabetic) are treated the illness of diabetes in different ways. Some are able to manage the symptoms of diabetes through a low-sugar diet, exercise and/or weight reduction. In many instances, a diabetic must take one or more daily injections of medication (e.g., insulin) to manage the symptoms of diabetes.

For some diabetic who takes one or more daily injections of medication, it can be difficult to remember if a particular dose on a particular day has been taken. This is especially true in the case where a diabetic is required to take multiple injects of medication each day. As a result, for some diabetics, the problem of taking too few or too many injections per day does exist. A non-uniform regimen of medication injections can and does have an adverse affect on the health of diabetics.

Therefore, a syringe case configured for aiding prescribed delivery of a syringe-injected medication such as insulin is useful and advantageous.

### SUMMARY OF THE DISCLOSURE

Embodiments of the present invention are specifically configured for aiding in prescribed delivery of a syringe-injected medication. In doing so, embodiments of the present invention advantageously reduce the potential for a patient taking too much medication or missing a dose of medication due to forgetfulness.

In one embodiment of the present invention, a syringe case comprises a first structural body and a second structural body. The first structural body includes a syringe retaining structure and a medication container retaining structure. The syringe retaining structure and the medication container retaining structure reside within an interior space of the first structural body. The syringe retaining structure is configured for holding a plurality of syringe such that a longitudinal axis of each syringe extends generally parallel with a longitudinal reference axis. The medication container retaining structure is configured for holding a medication container in a manner such that a longitudinal axis of the medication container extends generally perpendicular to the longitudinal reference axis. The second structural body is movably attached to the first structural body for enabling the structural bodies to be moved between an orientation providing a generally enclosed space between the structural bodies and an orientation exposing the interior space of the first structural body.

In another embodiment of the present invention, a syringe case comprises a first tray and a second tray. The first tray includes a plurality of spaced apart syringe retainers and a medication container retaining structure. The plurality of spaced apart syringe retainers of the first tray and the medication container retaining structure of the first tray reside within an interior space of the first tray. Adjacent ones of the syringe retainers of the first tray are each configured for holding a respective syringe such that a longitudinal axis of each syringe extends generally parallel with a longitudinal reference axis. The medication container retaining structure of the first tray is configured for holding a first medication container in a manner such that such that a longitudinal axis of the first medication container extends generally perpendicular to the longitudinal reference axis. The second tray includes a plurality of spaced apart syringe retainers and a medication container retaining structure. The plurality of spaced apart syringe retainers of the second tray and the medication container retaining structure of the second tray reside within an interior space of the second tray. Adjacent ones of the syringe retainers of the second tray are each configured for holding a respective syringe such that a longitudinal axis of each syringe extends generally parallel with the longitudinal reference axis. The medication container retaining structure of the second tray is configured for holding a second medication container in a manner such that a longitudinal axis of the second medication container extends generally perpendicular to the longitudinal reference axis. The second tray is movably attached to the first tray for enabling the trays to be moved between an orientation providing a generally enclosed space between the trays and an orientation exposing the interior space of each one of the trays.

In another embodiment of the present invention, a syringe case comprises a first tray, a second tray and a hinge. The first tray includes a plurality of spaced apart syringe retainers and two spaced part medication container retainers. Adjacent ones of the syringe retainers of the first tray are each configured for holding a respective syringe such that a longitudinal axis of each syringe extends generally parallel with a longitudinal reference axis. The two spaced apart medication container retainers of the first tray are jointly configured for holding a first medication container in a manner such that a longitudinal axis of the first medication container extends generally perpendicular with respect to the longitudinal reference axis. The second tray includes a plurality of spaced apart syringe retainers and two spaced apart medication container retainers. Adjacent ones of the syringe retainers of the second tray are each configured for holding a respective syringe such that a longitudinal axis of each syringe extends generally parallel with a longitudinal reference axis. The two spaced apart medication container retainers of the second tray are jointly configured for holding a second medication container retainer in a manner such that a longitudinal axis of the second medication container extends generally perpendicular to the longitudinal reference axis. The hinge is connected between the first tray and the second tray for enabling the trays to move between a closed orientation and an open orientation with respect to each other.

Turning now to specific aspects of the present invention, in at least one embodiment, the medication container retaining structure of the first tray is adjacent an end wall of the first tray, the medication container retaining structure of the second tray is adjacent an end wall of the second tray and the end wall of the first tray and the end wall of the second tray are positioned at opposite ends of the trays.

In at least one embodiment of the present invention, a plurality of day-designating markings are provided on each one of the trays, each one of the day-designating markings designates a particular day of the week and each one of the day-designating markings is positioned such that it is adjacent to a respective mounted syringe location and visible when the respective mounted syringe is engaged with a respective one of the spaced apart syringe retainers of the respective one of the trays.

In at least one embodiment of the present invention, a plurality of time-of-day designating markings are provided on each one of aid trays, each one of the time-of-day designating markings designates a particular time-of-day and each one of the time-of-day designating markings is positioned such that it is adjacent to a respective mounted syringe location and visible when the respective mounted syringe is engaged with a respective one of the spaced apart syringe retainers of the respective one of the trays.

In at least one embodiment of the present invention, the spaced apart syringe retainers of the trays each include opposing flexible arms configured for providing compressive force on the respective syringe

In at least one embodiment of the present invention, the medication container retainer of each tray each includes opposing flexible arms configured for providing compressive force on the respective medication container.

In at least one embodiment of the present invention, the medication container retainer of a particular tray is adjacent an end wall of the particular tray and all of the spaced apart syringe retainers of the particular tray are positioned between a lateral centerline of the particular tray and the medication container retaining structure.

These and other objects, embodiments, advantages and/or distinctions of the present invention will become readily apparent upon further review of the following specification, associated drawings and appended claims.

### BRIEF DESCRIPTION OF THE DRAWINGS

FIG. 1 is a perspective view of a syringe case in accordance with the present invention, which is in a fully open orientation.

FIG. 2 is a cross sectional view taken along the line 2-2 in FIG. 1.

FIG. 3 is a top view of the syringe case of FIG. 1.

### DETAILED DESCRIPTION OF THE DRAWING FIGURES

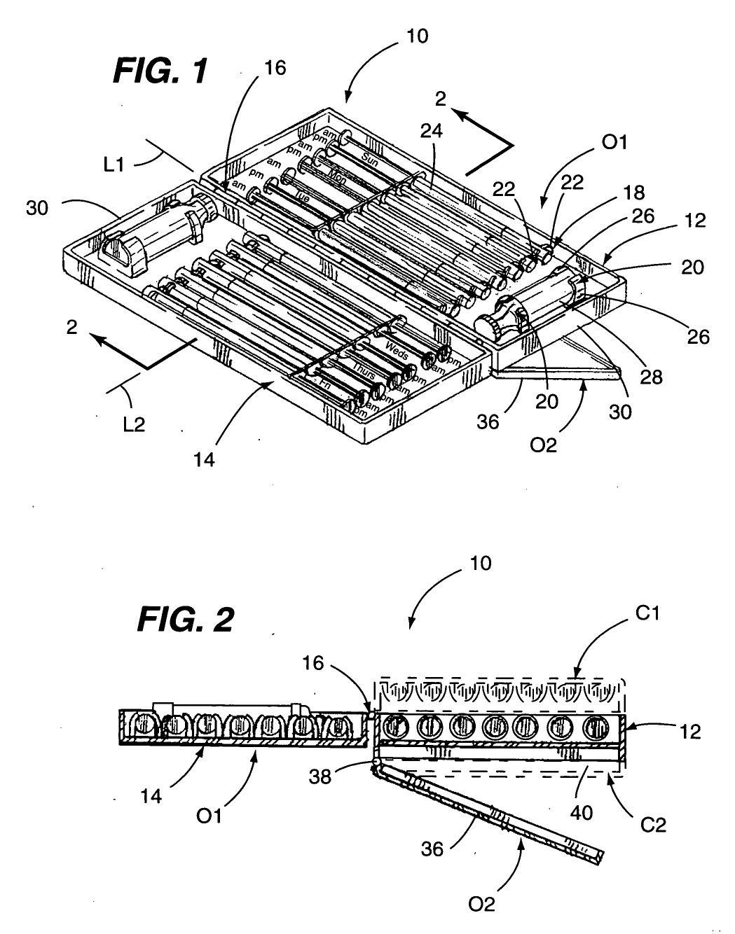

FIGS. 1-3 each show specific aspects of an embodiment of a syringe case in accordance with the present invention, which is referred to herein as the syringe case 10. The syringe case 10 includes a first tray 12, a second tray 14 and a hinge 16. The first tray 12 and the second tray 14 are examples of structural bodies in accordance with the present invention. A tray is defined herein to be a structural body with a raised edge or raised edges around at least a portion of its perimeter. A generally flat lid is another example of a structural body in accordance with the present invention, which may be used in place of one of the trays (12, 14).

The hinge 16 is connected between the first tray 12 and the second tray 14 for enabling the trays (12, 14) to move with respect to each other between a closed orientation C1 (shown in FIG. 2) and an open orientation O1 (shown in FIGS. 1-3). When the trays (12, 14) are in the closed orientation C1, a generally enclosed space 17 (FIG. 2) is provided between the trays (12, 14). When the trays (12, 14) are in the open orientation O1, an interior space of each tray (12, 14) is exposed, thereby enabling access to contents of the enclosed space 17.

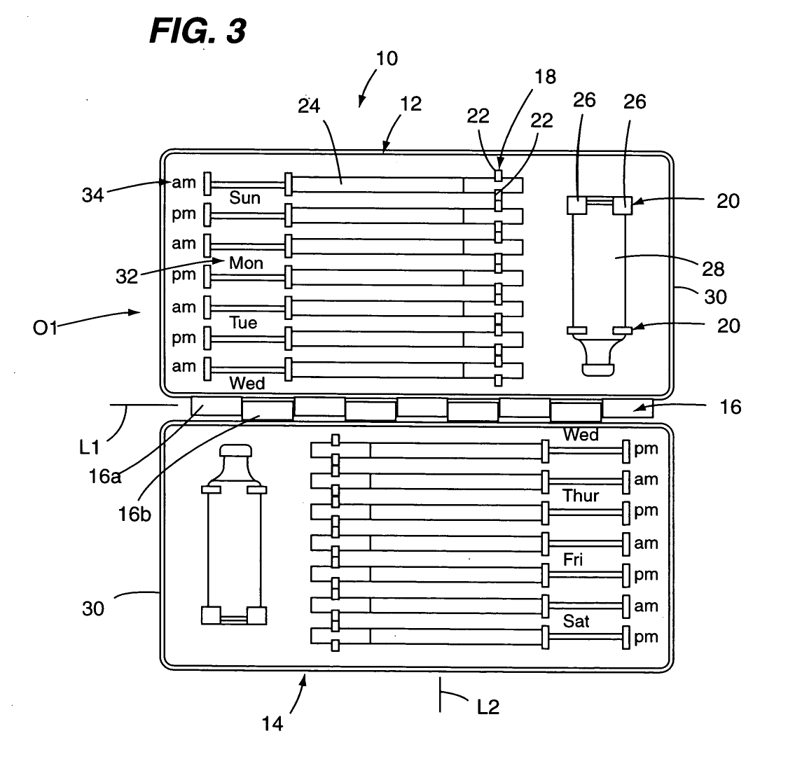

Preferably, but not necessarily, the hinge 16 is jointly integrated with the first tray 12 and a second tray 14. As depicted in FIGS. 1 and 3, the hinge 16 includes a first set of hinge lugs 16a attached to the first tray 12 and a second set of hinge lugs 16b attached to the second tray. Through a means for engaging the hinge lugs (16a, 16b) such as, for example, a rod extending adjacent ones of the hinge lugs (16a, 16b), the hinge 16 permits movement of the trays (12, 14) between the open and closed orientations (O1, C1).

Each one of the trays (12, 14) includes a plurality of spaced apart syringe retainers 18 and two spaced part medication container retainers 20. Preferably, but necessarily, each tray includes enough syringes retainers for holding seven (7) syringes and at least one medication container. Each one of the syringe retainers 18 includes opposing flexible arms 22 configured for exerting compressive force on a respective engaged syringe 24 for securing the respective engaged syringe 24 in a relatively fixed position within the respective one of the trays (12, 14). Each one of the medication container retainers 20 includes opposing flexible arms 26 configured for exerting compressive force on a respective engaged medication container 28 for securing the respective engaged medication container 28 in a relatively fixed position within the respective one of the trays (12, 14). The syringe retainers 18 and the medication container retainers 20 are respective examples of a syringe retaining structure and a medication container retaining structure in accordance with the present invention. In view of the disclosures made herein a skilled person will appreciate different configurations of syringe retaining structures and medication container retaining structures in accordance with the present invention.

Adjacent ones of the syringe retainers 18 are each configured for holding a respective syringe such that a longitudinal axis of each syringe 24 extends generally parallel with a longitudinal reference axis L1. The medication container retainers 20 are jointly configured for holding the medication container 28 in a manner such that a longitudinal axis of the medication container 28 extends generally perpendicular with respect to the longitudinal reference axis L1. Preferably, but not necessarily, the medication delivery container of a particular one of the tray (12, 14) resides adjacent an end of all of the syringes 24 mounted within the particular one of the trays (12, 14). For example, in the depicted embodiment, the medication container retainers 20 of each one of the trays (12, 14) are adjacent an end wall 30 of the respective one of the trays (12, 14) and the syringe retainers 18 of each one of the trays (12, 14) are positioned between a lateral centerline L2 of the respective tray (12, 14) and the medication container retainers 20 of the respective tray (12, 14). Furthermore, preferably, but not necessarily, the medication container retainers 18 of the first tray 12 and the medication container retainers 18 of the second tray 14 positioned adjacent opposite end walls of the trays (12, 14).

A plurality of day-designating markings 32 is provided on each one of the trays (12, 14). Each one of the day-designating markings 32 designates a particular day of the week (e.g., abbreviations for names of the days of the week). Each one of the day-designating markings 32 is positioned such that it is adjacent to a respective mounted syringe location (i.e., a location where a syringe is mounted via the syringe retainers 18) and is visible when the respective syringe 24 (i.e., a mounted syringe) is engaged with the respective syringe retainers 18. A plurality of time-of-day designating markings 34 provided on each one of the trays (12, 14). Each one of the time-of-day designating markings 34 designates a particular time-of-day (e.g., abbreviations for morning and afternoon). Each one of the time-of-day designating markings 34 is positioned such that it is adjacent to the respective mounted syringe location and is visible when the respective mounted syringe is engaged with a respective one of the spaced apart syringe retainers 18.

A storage compartment lid 36 is pivotably attached to the first tray 12 via a hinge 38. The storage compartment lid 36 is movable about the hinge 38 between an open orientation O2 (i.e., depicted as a partially open orientation) and a closed orientation C2. When the storage compartment lid 36 is in the closed orientation C2, a generally enclosed storage compartment 40, which is generally opposite the enclosed space 17 provided between the trays (12, 14), is provided between the first tray 12 and the storage compartment lid 36. When the storage compartment lid 36 is in the open orientation O1, access within the storage compartment 40 is provided.

In the preceding detailed description, reference has been made to the accompanying drawings that form a part hereof, and in which are shown by way of illustration specific embodiments in which the present invention may be practiced. These embodiments, and certain variants thereof, have been described in sufficient detail to enable those skilled in the art to practice embodiments of the present invention. It is to be understood that other suitable embodiments may be utilized and that logical, mechanical, chemical and electrical changes may be made without departing from the spirit or scope of such inventive disclosures. To avoid unnecessary detail, the description omits certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A syringe case, comprising:
a first structural body including syringe retaining structure, wherein said syringe retaining structure resides within an interior space of the first structural body, wherein said syringe retaining structure is configured for holding a plurality of syringes such that a longitudinal axis of each syringe extends generally parallel with a longitudinal reference axis;
a second structural body movably attached to the first structural body for enabling said structural bodies to be moved between an orientation providing a generally enclosed space between said structural bodies and an orientation exposing the interior space of the first structural body;
a plurality of day-designating markings provided on the first structural body, wherein each one of said day-designating markings designates a particular day of the week and wherein each one of said day-designating markings is positioned such that it is adjacent to a respective mounted syringe location and visible when the respective mounted syringe is engaged with said syringe retaining structure; and
a plurality of time-of-day designating markings provided on the first structural body, wherein each one of said time-of-day designating markings designates a particular time-of-day and wherein each one of said time-of-day designating markings is positioned such that it is adjacent to a respective mounted syringe location and visible when the respective mounted syringe is engaged with said syringe retaining structure.

2. The syringe case of claim 1 wherein:
the first structural body includes medication container retaining structure;
said medication container retaining structure is configured for holding a medication container in a manner such that a longitudinal axis of the medication container extends generally perpendicular to the longitudinal reference axis.

3. The syringe case of claim 2 wherein:
said medication container retaining structure is adjacent an end wall of the first structural body; and
said syringe retaining structure is positioned between a lateral centerline of the first structural body and said medication container retaining structure.

4. The syringe case of claim 3 wherein:
said syringe mounting structure includes a plurality of spaced apart syringe retainers that each include opposing flexible arms configured for providing compressive force on the respective syringe; and
said medication container retaining structure includes opposing flexible arms configured for providing compressive force on the medication container.

5. The syringe case of claim 4 wherein:
said medication container retaining structure is adjacent an end wall of the first structural body; and
said syringe retaining structure is positioned between a lateral centerline of the first structural body and said medication container retaining structure.

6. The syringe case of claim 1 wherein:
said syringe mounting structure includes a plurality of spaced apart syringe retainers that each include opposing flexible arms configured for providing compressive force on the respective syringe.

7. A syringe case, comprising:
a first tray including a plurality of spaced apart syringe retainers and a medication container retaining structure, wherein the plurality of spaced apart syringe retainers of the first tray and the medication container retaining structure of the first tray reside within an interior space of the first tray, wherein adjacent ones of said syringe retainers of the first tray are each configured for holding a respective syringe such that a longitudinal axis of each syringe extends generally parallel with a longitudinal reference axis and wherein the medication container retaining structure of the first tray is configured for holding a first medication container in a manner such that such that a longitudinal axis of the first medication container extends generally perpendicular to the longitudinal reference axis; and
a second tray including a plurality of spaced apart syringe retainers and a medication container retaining structure, wherein the plurality of spaced apart syringe retainers of the second tray and the medication container retaining structure of the second tray reside within an interior space of the second tray, wherein adjacent ones of said syringe retainers of the second tray are each configured for holding a respective syringe such that a longitudinal axis of each syringe extends generally parallel with a longitudinal reference axis, wherein the medication container retaining structure of the second tray is configured for holding a second medication container in a manner such that a longitudinal axis of the second medication container extends generally perpendicular to the longitudinal reference axis and wherein the second tray is movably attached to the first tray for enabling said trays to be moved between an orientation providing a generally enclosed space between said trays and an orientation exposing the interior space of each one of said trays.

8. The syringe case of claim 7 wherein:
the medication container retaining structure of the first tray is adjacent an end wall of the first tray;
the medication container retaining structure of the second tray is adjacent an end wall of the second tray; and
the end wall of the first tray and the end wall of the second tray are positioned at opposite ends of said trays.

9. The syringe case of claim 7, further comprising:
a plurality of day-designating markings provided on each one of said trays, wherein each one of said day-designating markings designates a particular day of the week and wherein each one of said day-designating markings is positioned such that it is adjacent to a respective mounted syringe location and visible when the respective mounted syringe is engaged with a respective one of said spaced apart syringe retainers of the respective one of said trays.

10. The syringe case of claim 9, further comprising:
a plurality of time-of-day designating markings provided on each one of aid trays, wherein each one of said time-of-day designating markings designates a particular time-of-day and wherein each one of said time-of-day designating markings is positioned such that it is adjacent to a respective mounted syringe location and visible when the respective mounted syringe is engaged with a respective one of said spaced apart syringe retainers of the respective one of said trays.

11. The syringe case of claim 7 wherein:
said spaced apart syringe retainers of said trays each include opposing flexible arms configured for providing compressive force on the respective mounted syringe; and
the medication container retaining structure of said trays each include opposing flexible arms configured for providing compressive force on the respective medication container.

12. The syringe case of claim 11 wherein:
the medication container retaining structure of the first tray is adjacent an end wall of the first tray;
the medication container retaining structure of the second tray is adjacent an end wall of the first second; and
the end wall of the first tray the end wall of the second tray are positioned at opposite ends of said trays.

13. The syringe case of claim 12, further comprising:
a plurality of day-designating markings provided on each one of said trays, wherein each one of said day-designating markings designates a particular day of the week and wherein each one of said day-designating markings is positioned such that it is adjacent to a respective mounted syringe location and visible when the respective mounted syringe is engaged with a respective one of said spaced apart syringe retainers of the respective one of said trays; and
a plurality of time-of-day designating markings provided on each one of aid trays, wherein each one of said time-of-day designating markings designates a particular time-of-day and wherein each one of said time-of-day designating markings is positioned such that it is adjacent to a respective mounted syringe location and visible when the respective mounted syringe is engaged with a respective one of said spaced apart syringe retainers of the respective one of said trays.

14. A syringe case, comprising:
a first tray including a plurality of spaced apart syringe retainers and two spaced part medication container retainers, wherein adjacent ones of said syringe retainers of the first tray are each configured for holding a respective syringe such that a longitudinal axis of each syringe extends generally parallel with a longitudinal reference axis and wherein said two spaced apart medication container retainers of the first tray are jointly configured for holding a first medication container in a manner such that a longitudinal axis of the first medication container extends generally perpendicular with respect to the longitudinal reference axis;
a second tray including a plurality of spaced apart syringe retainers and two spaced apart medication container retainers, wherein adjacent ones of said syringe retainers of the second tray are each configured for holding a respective syringe such that a longitudinal axis of each syringe extends generally parallel with a longitudinal reference axis and wherein said two spaced apart medication container retainers of the second tray are jointly configured for holding a second medication container retainer in a manner such that a longitudinal axis of the second medication container extends generally perpendicular to the longitudinal reference axis;
a hinge connected between the first tray and the second tray for enabling said trays to move between a closed orientation and an open orientation with respect to each other.

15. The syringe case of claim 14 wherein:
said medication container retainers of the first tray are adjacent an end wall of the first tray;
said medication container retainers of the second tray are adjacent an end wall of the second tray; and
the end wall of the first tray and the end wall of the second tray are positioned at opposite ends of said trays.

16. The syringe case of claim 15, further comprising:
a plurality of day-designating markings provided on each one of said trays, wherein each one of said day-designating markings designates a particular day of the week and wherein each one of said day-designating markings is positioned such that it is adjacent to a respective mounted syringe location and visible when the respective mounted syringe is engaged with a respective one of said spaced apart syringe retainers of the respective one of said trays.

17. The syringe case of claim 16, further comprising:
a plurality of time-of-day designating markings provided on each one of aid trays, wherein each one of said time-of-day designating markings designates a particular time-of-day and wherein each one of said time-of-day designating markings is positioned such that it is adjacent to a respective mounted syringe location and visible when the respective mounted syringe is engaged with a respective one of said spaced apart syringe retainers of the respective one of said trays.

18. The syringe case of claim 17 wherein:
said spaced apart syringe retainers of said trays each include opposing flexible arms configured for providing compressive force on the respective syringe; and
said medication container retainers of said trays each include opposing flexible arms configured for providing compressive force on the respective medication container.
